(19) United States
(12) Patent Application Publication
(10) Pub. No.: US 2019/0103180 A1
(43) Pub. Date: Apr. 4, 2019

(54) Women Fertility and Life Event Tracker and Planner

(71) Applicant: Hilin Life Products, Inc., Newark, NJ (US)

(72) Inventors: Helen Denise, Paramus, NJ (US); Alexander Axenov, Brooklyn, NY (US)

(73) Assignee: Hilin Life Products, Inc., Newark, NJ (US)

(21) Appl. No.: 16/150,837

(22) Filed: Oct. 3, 2018

Related U.S. Application Data
(60) Provisional application No. 62/567,325, filed on Oct. 3, 2017.

(51) Int. Cl. G16H 20/10; G16H 50/30; G06F 3/0482

(52) U.S. Cl. CPC G16H 20/10; G16H 80/00; G06F 3/0482; G16H 50/30

(57) ABSTRACT

A pregnancy planner for personal electronic devices wherein a user's device receives one or more of health elements, each of which is assigned a numeric value, and wherein a total fertility status indicating the probability of a subject to conceive is determined by summing the values of the health elements. In certain embodiments, the disclosed platform includes a planner for inputting and tracking life events.

### FIELD OF INVENTION

The subject matter of the present disclosure generally relates to planning, and more particularly relates to a life event tracker and planner.

### BACKGROUND

Previous systems for detecting ovulation via saliva have been disclosed. The subject matter of U.S. Patent Publication No. 2011/0282247, entitled “Novel Ovulation Predictor Device” and filed Mar. 31, 2011, is hereby incorporated herein in its entirety.

### BRIEF DESCRIPTION

Disclosed is a system and method for tracking and monitoring sexual health and other personal information, which may be employed on a user's personal electronic device (PED). In certain embodiments, the system and method are designed for use with an ovulation monitoring test by which a user's ovulation cycle may be monitored.

### BRIEF DESCRIPTION OF THE DRAWINGS

FIG. 1 depicts a user interface of an embodiment as presented on a user's PED.

FIG. 2 depicts an avatar as displayed over the user interface of the embodiment of FIG. 1.

FIG. 3 depicts a menu overlay as displayed over the user interface of the embodiment of FIG. 1.

FIG. 4 depicts a detailed settings page for the embodiment of FIG. 1.

FIG. 5 depicts a menstruation and ovulation tracking page for the embodiment of FIG. 1.

FIG. 6 depicts a calendar and life event tracking page for the embodiment of FIG. 1.

FIG. 7 depicts a data entry form for the life event tracking page of FIG. 6.

### DETAILED DESCRIPTION

In an exemplary embodiment now described, a user enters several elements of sexual health information. After a period of time, automated recommendation information is provided to the user. The user can further track statistics related to or derived from the entered sexual health information.

In the exemplary embodiment, the system and method are performed using a Personal Electronic Device (PED) such as smartphone or tablet computer. One of ordinary skill will recognize the disclosed system and method may be performed using other electronic devices. For the purposes of this disclosure, the system and method are referred to as the App as installed and used with the user's PED.

In the exemplary embodiment, the user is prompted on launching the App to sign in if they have an account or create one if they do not have an existing account.

The user may be prompted to enter information pertaining to them, including, without limitation:
First Name
Last Name
Date of Birth
E-mail Address
Password
Address
Profession
Education
Marital Status

The user may also be prompted during the sign up process to accept a set of terms & conditions. Further, the user may be provided an option to restore a lost password.

Optionally, the user may be permitted to sign in with another social media service, such as Google, Facebook, Pinterest, Twitter etc. If using another social media service, upon signing the user may be prompted to enter a privacy password, as well as to accept or decline additional functions such as posting on that social media platform, etc. In certain cases, the user is permitted to send and receive messages to other users using the social media platform. If the user's PED is not connected with the internet when in use, data can be stored locally while the App is in an “offline” and can then transmit data once the App is connected again.

Tutorial Mode

Optionally, the App may present a tutorial mode. For the first run or re-initiation, from a side menu, the user may be asked whether or not they wish to conceive. The tutorial mode may then run through various screens, highlighting areas and explaining the purpose of the various elements. The App may prompt the user to purchase an ovulation monitoring device.

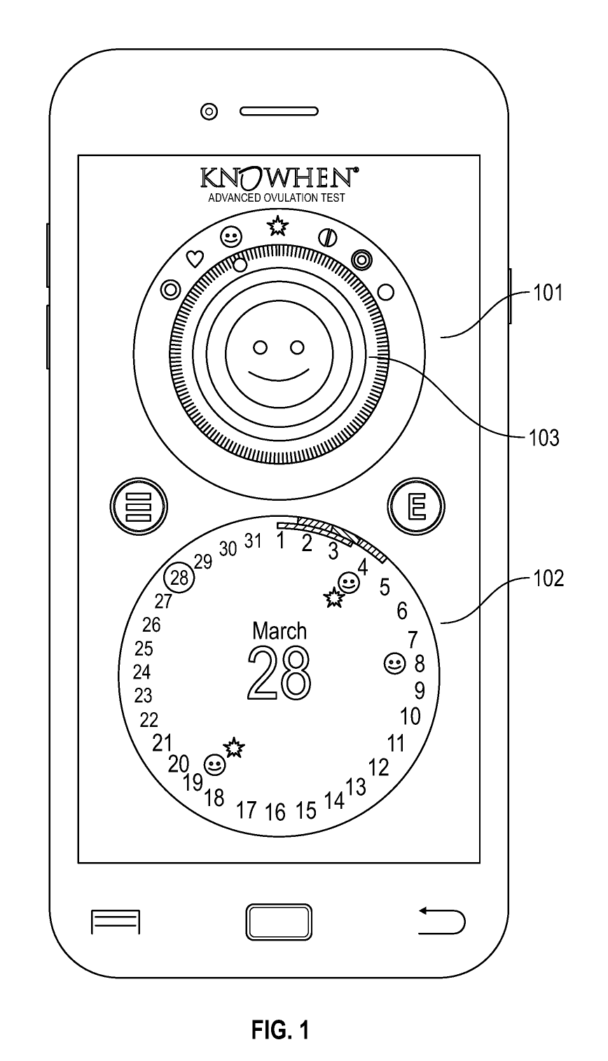

FIG. 1 depicts the Main Screen of a first embodiment. There is an Input Area 101 and Calendar View Area 102.

Input Area

In the Input Area, the user can enter personal data. There is a rotating dial 103 that rotates with tick steps. The user places their finger onto a selector, and rotates the dial by dragging their finger against the input screen around the periphery of the dial, whereby the selector rotates until the user reaches a desired input. An indication of the data input type which the selector is against is displayed in the center of the dial. The user can enter at least the following data:
a. Mood—May be indicated as Happy|OK|Sad
b. Ovulation—May be indicated as Yes|Maybe|No
c. Menstruation—May be indicated as No|Normal|Weak|Strong
d. Intercourse—May be indicated as No|1|2+
e. Orgasm—May be indicated as No|Yes
f. Contraceptives—May be indicated as No|Chemical|Physical
g. Stressful Event—May be indicated as No|Stressful|very stressful
h. Temperature—May be included as Low/Normal/High, or by degrees Celsius or Fahrenheit
i. Blood pressure—May be included as Low/Normal/High, or by numbers

Each selection provides an input using the inside of rotating dial 103. By tapping on the center of rotating dial 103 the user can switch the type of the answer. Optionally, each selection can involve a maximum of 3-4 options. For example, the Mood input may have three potential states of: Default state—Happy, 1 tap=ok , 2nd tap=sad. Optionally, certain input options can be disabled in a Settings page. Therefore, to enter a data input for mood, the user would press their finger against the dial selector, moving it until it is next to the indicator for mood (in the case of FIG. 1, a smiley face). The user then releases and taps the center of the dial to change between the mood indications. For instance, by default it may be a smiley face but the user can then tap the center of the dial to change the data input to a frowny face.

Optionally, if one or more of the optional input items are turned off in the settings, then the associated icon is put in a disabled state and the rotator selection mechanism will skip or jump through that selection to the next available input item, provided there is no lock on rotation.

Each selected Icon should indicates that an active selection is being made through a highlighting state.

Calendar View Area

The “Eva” avatar is an animated avatar that can deliver information, alerts and reminders to the user, providing an interactive user experience.

In the exemplary embodiment, the Calendar View Area has the following features:
a. The Calendar View Area contains a full month in a circle, which can optionally be expanded with a sliding out animation.
b. The 3 o'clock provides the current date and may be highlighted through a selector.
c. Swiping Left or Right displays a different calendar month, so that input history may be easily reviewed.
d. Swiping Up or Down changes the displayed day (For example, Up by −1 day and Down by +1 day).
e. Dragging on the side of the wheel rotates dates.
f. If another day was selected, than “today's date” is provided extra identification. (for example, by a spiral around “today's date” or neon dot beside)
g. The calendar always displays a red line (with an icon from main rotator) besides menstruation dates
h. The Calendar also always display a blue line (with icon from main rotator) besides ovulation dates
i. Depending on the position of the main rotor (for options Mood, Intercourse, Orgasm, and Contraception) it displays a corresponding selection on the calendar at corresponding dates where a selection was made.
   i. When the Rotator is on the Mood icon, indicators (optionally displayed in 3 colors) are displayed on the calendar circle at corresponding dates where a selection was made.
   ii. When the Rotator is on the Ovulation icon, an ovulation bar is displayed on the calendar circle.
   iii. When the Rotator is on the Menstruation Icon, a menstruation bar is displayed on the calendar circle.
   iv. When the Rotator is on the Sex Icon, a Sex icon is displayed on the calendar circle at corresponding dates where an input was made.
   v. When the Rotator is on the Orgasm icon, a Orgasm icon is displayed on the calendar circle at the corresponding date where an input was made, in addition to the sex icon being displayed.
   vi. When the Rotator is on the Contraception icon, a Contraception icon is displayed, indicating the type of contraception used, in addition to the sex icon being displayed.
   vii. When the Rotator is on the Temperature icon, a Temperature icon is displayed, indicating the temperature in C and/or F.
   viii. When the Rotator is on the Blood Pressure icon, a Blood Pressure icon is displayed, indicating the Blood Pressure in numbers.
j. On top of the Calendar a face of the Eva appears when the Eva avatar provides a comment or feedback.

Eva Button

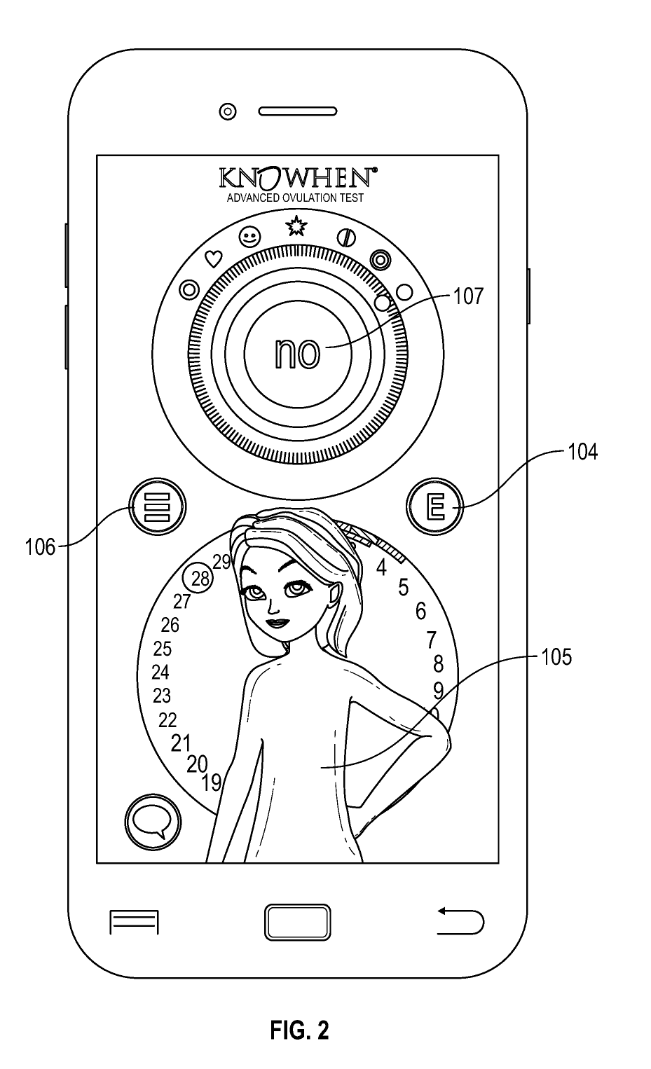

With reference to FIG. 2, the Eva avatar 104 may provide feedback to the user depending on their input data. For instance, in certain embodiments, if the data provided by the user indicates that the user is ovulating, Eva may advise the user that this may be the case for pregnancy planning purposes.

In the embodiment, the Eva Button 105 has the following functionality:
a. The button is highlighted when the Eva Avatar has feedback—which may be a “today tip” for that day, regular analytics or advice.
b. The button is not highlighted if the Eva Avatar does not have feedback. Nonetheless, if the Eva button is pressed in this scenario the Avatar provides a random phrase or useful tip.
c. Optionally, voice clips and lips syncs may be downloaded or pushed from a server
d. A “cloud button” duplicates the voice text as a Text pop-up in the User Interface (UI).

Side Menu

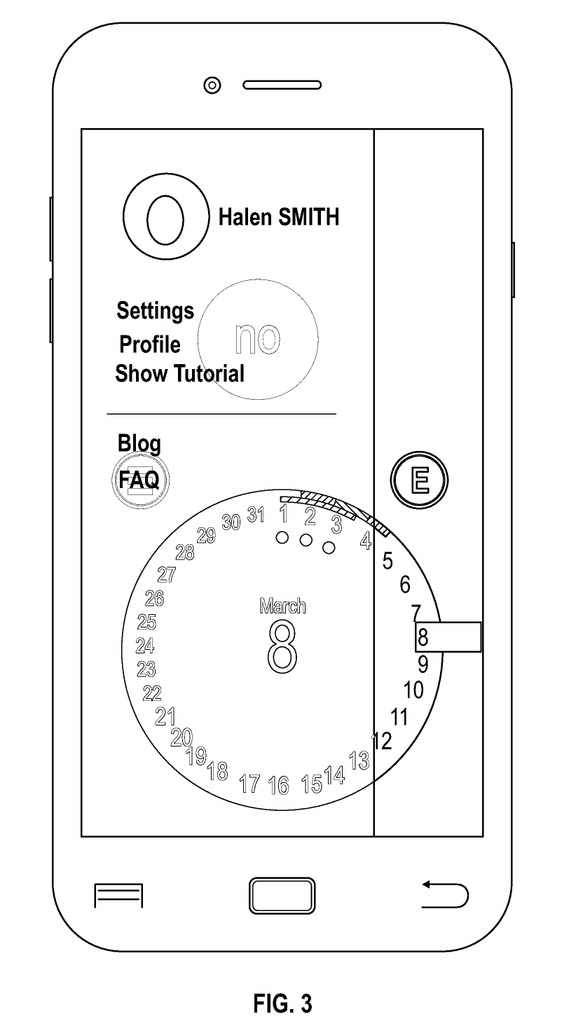

A side menu, accessed via menu button 106, and depicted in FIG. 3, displays the following information:
A profile picture from social media
The name of the person
A settings button
A profile Button
A link to the website blog
A link to a FAQ page
A link to a Fertility Game
A link to Last News
A link to Video Stories

Additional buttons and links may be provided.

Settings Screen

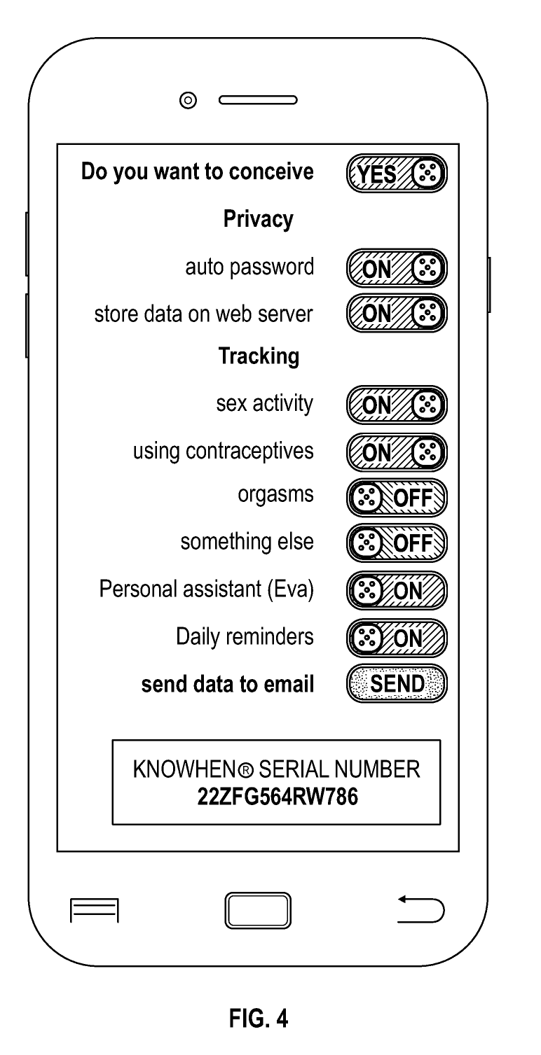

A setting screen, depicted in FIG. 4, may provide additional functionality. For instance, it can solicit a rating if the App has been opened more than three times. Also, the user can set confidentiality settings, for example the user may select to login automatically upon launching the App or may optionally require that their password be entered each time.

Analytics

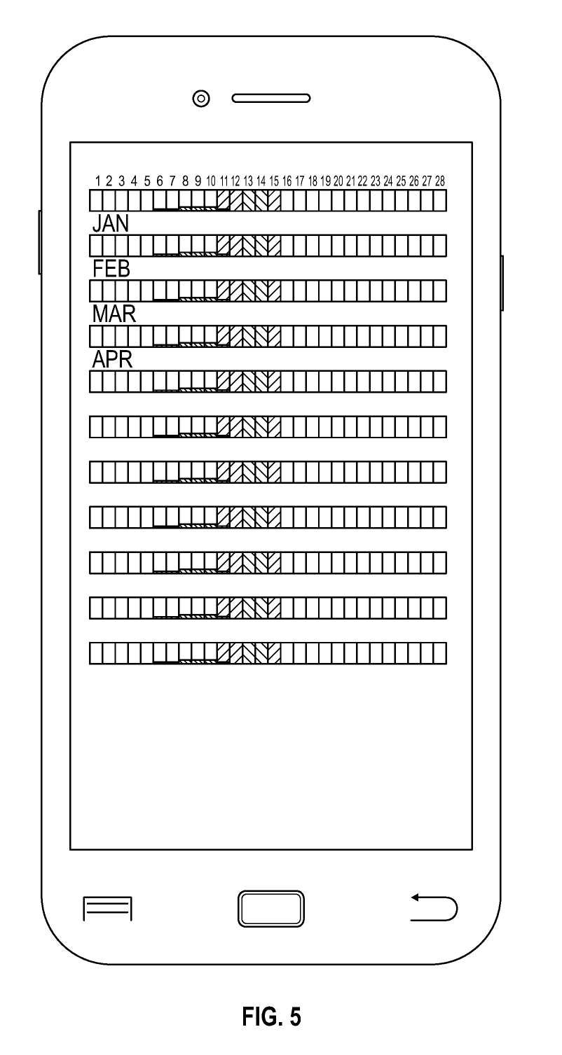

The App may provide the user analytics based on entered data. In the exemplary embodiment, after a period of collecting information (e.g., 2 months), the App may provide predictions of the user's Menstruation and Ovulation days. A depiction of certain analytics as provided to the user is depicted in FIG. 5.

Server Side Analytics

A server may perform numerous functions, such as tracking:
Unique users
Settings choices (for optional tracking entries)
Microscope purchases
result submissions per user
Eva usage
Eva text speech
Past record editing
Data export
Online/offline usage

The following notifications may be locally generated or pushed from the server:
A reminder to the user to submit ovulation test results for the day
If analytics something detects
Each 3 days a reminder to purchase microscope
Notices regarding special promotions (ex. sales discounts), Holidays, birthday greetings.
Yearly Reports

The server has the ability to perform the following functions:
Authorize users (registration and further authorization) either via standalone processes or through other online platforms
Password recovery
Store/Retrieve/Edit user input
Sync user input in case if device was offline and then become online
Register Physical device against user through unique Serial Number (SN)
Send Push Notifications
Send user data as export to email
Send subscription emails to all user base
Ability to unsubscribe from mailing list

An Admin Control Panel allows an admin to:
View statistics for, optionally:
   All users
   Active users
   Registered with SN users
Perform HTML file capture for emailing
Perform push notifications (title/body)

Other Features

In the exemplary embodiment, the App has the following additional features:
1. User-User communication—there is an in-app forum and/or user-to-user chat feature for communication among users.
2. User-Doctor communication—a user can securely and privately communicate with their physician.
3. Advanced analytics—on the server-side, anonymized granular input data may be analyzed (for example, across all users it can be monitored various health metrics).
6. The App may include mini-games, lotteries and in-app Advertisements
7. Additional data tracking/analysis (ex. type of contraceptives, weight, pain, appetite etc . . . )
8. Eva animation, voice, phrases updates
9. AR Eva with microscope instructions (demo already available)

Tracking of Metrics

In certain embodiments, the user may register life events (ex. mood, menstruation, sex activities) every day in the App to analyze their general health conditions in addition to their ability to conceive. The App provides feedback messages.

Every woman has a unique physiology, so tracking her life events that can affect her ability to ovulate and conceive allows better planning and achievement of pregnancy. Tracking her health conditions can also help improve general overall health.

App messages may be given based on registered events combinations, for example and without limitation, such as:

1. A planned trip and menstruation days as calculated by the App.
2. A planned party and ovulation as calculated by the App.
3. A planned business meeting and extreme moods near ovulation
4. Changes due to continuing stress
5. Changes due to travel

Metrics that can be tracked in the App:

### 1. Life Events

Negative and positive stress
Birthdays
Weddings
Business Meetings
Family Events
Romantic Dates
Parties
Trips
Appointments
Exercises
Mood
Orgasms
Diary
Plans & Goals
Accomplishments
Business Notes
Pictures
   2. Menstrual cycle regularity

### 3. Birth Control use

4. Ovulation Cycle window
5. Ovulation cycle regularity

Mood

There are several aspects that can impact mood, including without limitation:

1. Physical condition (regular exercise, sex activity)
2. Contraceptive use—some people report negative mood changes, depressive symptoms, or mood swings while taking hormonal birth control, and they seem to get better when they stop taking it or switch to another method
3. Environmental conditions (problem at work, in family & relations)

Tracking Contraceptive Pharmaceutical Use

According to certain research, taking birth control pills may diminish fertility. It was found that women who were on a birth control pill routine had smaller ovaries than those who did not take birth control pills, particularly in the youngest of the subjects, between the ages of 19 and 29.9. Generally, studies indicate that fertility returns to normal levels within a couple months of stopping the pills usage.

Tracking Stress

Stress can negatively interfere with conception. That's because stress can affect the functioning of the hypothalamus—the gland in the brain that regulates appetite and emotions, as well as the hormones that influence ovaries to release eggs. An individual under stress may ovulate later in their cycle or not at all. Therefore, only trying to conceive sex around day 14 in a woman's cycle may cause her to miss an opportunity to conceive.

It's important to differentiate between constant and sudden stress. If an individual's stress level is high and fairly consistent, the body will likely acclimate to it and they will still ovulate each cycle. However sudden stress, such as an accident or a death in the family, can significantly impact with a woman's cycle and interfere with ovulation.

These effects vary from woman to woman. Some women find that even a trip out of town can delay ovulation. Others have found that even a severely traumatic incident didn't impact their cycle.

Positive stress can also affect a woman's cycle, causing her to ovulate later or not at all. For example, brides often report strange cycles because they're happily stressed about their weddings.

Tracking Basal Body Temperature

A user may confirm they indeed ovulated by tracking their basal body temperature. Generally a woman will see a sustained rise about a day after ovulation, lasting 12-16 days.

Delayed ovulation generally lengthens the entire cycle, as opposed to shortening the luteal phase—typically lasting 12-16 days after ovulation until the start of the next menstrual period. This is important because a short luteal phase has been linked to early miscarriage. Tracking basal body temperature information may therefore be useful in tracking and predicting ovulation.

Tracking Mood

During the luteal phase many women feel hot or feel anxiety, depression, irritability and mood swings. Women taking hormonal birth control may not experience these symptoms with the same severity or at all.

Ovulation Prediction

The App may determine, based on entered information, a predication of when the user is ovulating. This may alert the user when to engage in intercourse to maximize their chance of getting pregnant, or if they are trying to avoid a pregnancy, when to avoid intercourse. In an embodiment, the App incorporates the following entered elements in predicting ovulation:

Elements:

### 1. Sexual Activity (SA)

### 2. Mood (MD)

### 3. Menstruation (ME)

### 4. Ovulation Test Result (OV)

### 5. Orgasm (OR)

### 6. Contraceptive Use (CU)

### 7. Daily Activity (DA)

### 8. Body Temperature (BT)

### 9. Ovulation Prediction (OP)

### 10. Stress (ST)

Today Fertility Status (TFS) is an indicator of the probability of conceiving. In one embodiment, the TFS is expressed as a percentage, for example that ranges from 99% to 10%.

TFS=SA+MD+ME+OV+OR+CU+CC+BT+OP+ST

### Wherein, for SA:

If sexual activity in the last five days=+30

If no sexual activity in the last five days=−30

for OV:

Ovulation test indicates YES=+100

Ovulation test indicates MAYBE=+85

Ovulation test indicates NO=−100

for OP:

If the ovulation tracking indicates ovulation=+80

for ME:

high intensity menstruation (last wet day more than 5 days)=+35

low intensity menstruation (last wet day more than 5 days)=+35

no menstruation=0

for ST:

if a high stress event has been recorded in the last seven days=−30

if a low stress event has been recorded in the last seven days=−10

for OR:

if a orgasm has been recorded in the last seven days=+20

for CU:

if contraception has been recorded in the last thirty days=−90

for CC:

if travel has been recently made to a different climate=−15

for BT:

if temperature more than 36.7° C.=+50

for MD:

if mood has been marked as happy in the last seven days=+30

if mood has been marked as sad in the last seven days=−30

In certain embodiments, elements may be “stacked” or counted more than once, for instance if the user has multiple high stress events.

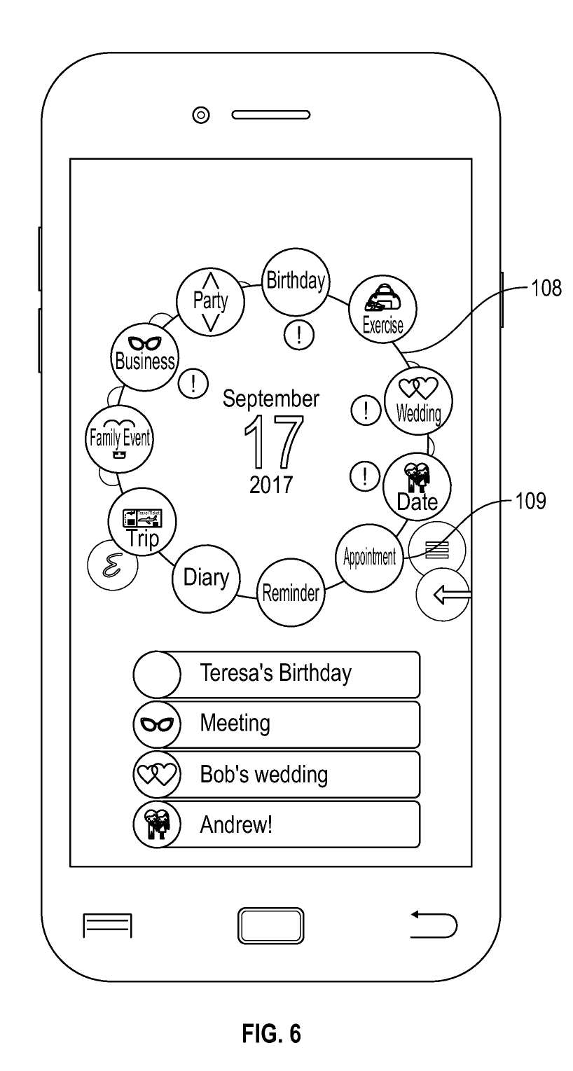

Certain embodiments incorporate a Planner, as shown in FIG. 6. The Planner screen covers the main screen after tapping the “planning” button 107. With reference to FIG. 6, the circle 108 of round buttons 109 represents events.

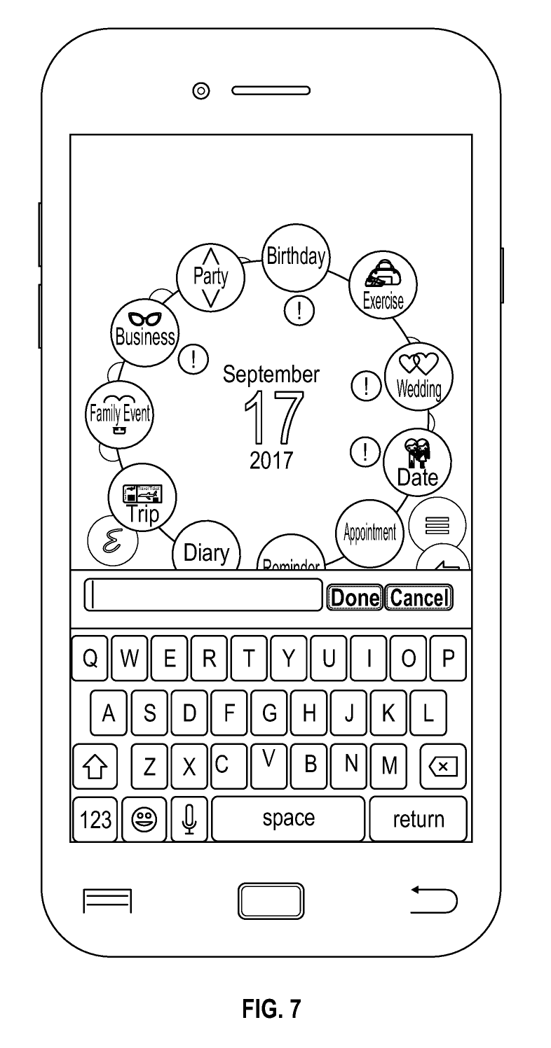

Tapping on any of the buttons 109 or dragging the buttons 109 to the center of circle 108 initiates pop-up text input area for user note input. After input is done a colored bar with text appears in the lower part of the planner screen as well as an exclamation mark icon near corresponding button, as shown in FIG. 7 The displayed color depends of type of event. Options for events include: yearly, monthly, weekly or “one time” event.

Tapping on a bar, as shown in FIG. 7, opens a pop-up text input area for editing the note or removing the text. If the user removes text from input window the bar disappears and the event is removed from calendar. Sliding the bar left or right removes it from the column Closing the planner screen saves the entered data in the main calendar database. Birthdays and wedding anniversaries may be automatically recorded for the next 5 years.

Events that may be included in the planner include:
Birthdays
Weddings
Business Meetings
Family Events
Dates
Parties
Location/trips
Appointments
Exercise
Entertainment/movies, sport, raising

Additional items that may be tracked include:

### 1. TO-DO's

Wants
Needs
Drafts

### 2. CONTACTS

Names
Family members
Friends
Addresses
Phones 3. MEMORIES
Likes
Times
Thoughts
Dreams
Views 4. SUMMARIES
Plans
Opportunities
Statements
Deals
Schedules
Goals
Responsibilities
Experiences 5. OBJECTIVES
Questions

What is claimed:

1. A pregnancy planner, comprising:
a personal electronic device (PED) having a display and a processor, and being configured to receive user inputs;
wherein the PED is configured to receive one or more of health elements including a Sexual Activity input, a Mood Input, a Menstruation Input, an Ovulation Test Input, an Orgasm Input, a Contraceptive Use Input, a Climate Change Input, a Body Temperature Input, an Ovulation Prediction Indicator and a Stress Input;
wherein each of the health elements is assigned a numeric value; and
wherein the processor is configured to determine a Total Fertility Status by summing the values of the health elements, wherein the Total Fertility Status is indicative of the probability of a subject for conceiving.

2. The pregnancy planner of claim 1, wherein the Sexual Activity Input is indicative of whether the subject has recently engaged in sexual intercourse.

3. The pregnancy planner of claim 1, wherein the Mood Input is indicative of the mood of the recent mood of the subject.

4. The pregnancy planner of claim 1, wherein the Menstruation Input is indicative of the intensity of menstruation the subject has recently experienced.

5. The pregnancy planner of claim 1, wherein the Ovulation Test Input is indicative of the results of an ovulation test administered on the subject.

6. The pregnancy planner of claim 1, wherein the Orgasm Input is indicative of whether the subject has recently experienced an orgasm.

7. The pregnancy planner of claim 1, wherein the Contraceptive Use Input is indicative of whether the subject is using contraceptives and, when so, what type.

8. The pregnancy planner of claim 1, wherein the Climate Change Input is indicative of whether the subject has recently traveled to a different climate.

9. The pregnancy planner of claim 1, wherein the Body Temperature Input is indicative of the subject's average body temperature.

10. The pregnancy planner of claim 1, wherein the Ovulation Prediction Indicator is indicative of whether ongoing month-over-month record of the menstruation cycles of the subject indicates the subject is ovulating.

11. The pregnancy planner of claim 1, wherein the Stress Input is indicative of the level of recent stress for the subject.

12. The pregnancy planner of claim 1, wherein presented on the display of the PED a planner screen.

13. The pregnancy planner of claim 12, wherein the planner screen includes a circular menu having a plurality of buttons, each button representing an event.

14. A method of pregnancy planning, comprising the steps of:
providing PED having a display and a processor, and being configured to receive user inputs;
receiving to the PED one or more of health elements including a Sexual Activity input, a Mood Input, a Menstruation Input, an Ovulation Test Input, an Orgasm Input, a Contraceptive Use Input, a Climate Change Input, a Body Temperature Input, an Ovulation Prediction Indicator and a Stress Input;
assigning each of the health elements a numeric value; and
determining a Total Fertility Status by summating the values of the health elements, wherein the Total Fertility Status is indicative of the probability of a subject for conceiving.

15. The pregnancy planner of claim 14, wherein the Menstruation Input is indicative of the intensity of menstruation the subject has recently experienced.

16. The pregnancy planner of claim 14, wherein the Ovulation Test Input is indicative of the results of an ovulation test administered on the subject.

17. The pregnancy planner of claim 14, wherein the Contraceptive Use Input is indicative of whether the subject is using contraceptives and, when so, what type.

18. The pregnancy planner of claim 14, wherein the Ovulation Prediction Indicator is indicative of whether ongoing month-over-month record of the menstruation cycles of the subject indicates the subject is ovulating.

19. The pregnancy planner of claim 14, wherein presented on the display of the PED a planner screen.

20. The pregnancy planner of claim 19, wherein the planner screen includes a circular menu having a plurality of buttons, each button representing an event, and receiving a selection of one the buttons and recording the associated event as having occurred.
